(19) United States
(12) Patent Application Publication
(10) Pub. No.: US 2011/0028773 A1
(43) Pub. Date: Feb. 3, 2011

(54) Deoxygenation of Bio-Oils and Other Compounds to Hydrocarbons in Supercritical Media

(76) Inventors: Bala Subramaniam, Lawrence, KS (US); Jackson W. Ford, Mobile, AL (US); Raghunath V. Chaudhari, Lawrence, KS (US)

Correspondence Address: STINSON MORRISON HECKER LLP;ATTN: PATENT GROUP, 1201 WALNUT STREET, SUITE 2900, KANSAS CITY, MO, 64106-2150, US

(21) Appl. No.: 12/846,407

(22) Filed: Jul. 29, 2010

Related U.S. Application Data
(60) Provisional application No. 61/229,625, filed on Jul. 29, 2009.

(51) Int. Cl. C07C 1/00

(52) U.S. Cl. 585/733

(57) ABSTRACT

A process for the complete deoxygenation of an oxygenate, especially those from bio-oils comprises forming a reaction mixture comprising the oxygenate, molecular hydrogen, and a hydrodeoxygenation catalyst in a solvent. The reaction mixture is maintained at a temperature that is 0.7 to 1.3 times the solvent critical temperature in absolute temperature units (K). Complete deoxygenation occurs via a hydrodeoxygenation pathway and a decarbonylation pathway.

### CROSS-REFERENCE TO RELATED APPLICATIONS

This application is based on and claims priority to U.S. Provisional Application Ser. No. 61/229,625 filed on Jul. 29, 2009 which is incorporated herein by reference.

### STATEMENT REGARDING FEDERALLY SPONSORED RESEARCH OR DEVELOPMENT

Not applicable.

### BACKGROUND OF THE INVENTION

Bio-oils derived from biomass pyrolysis show much promise as feedstocks for producing hydrocarbons that may be readily integrated as feeds into existing petroleum refineries as well as future biorefineries. Numerous upgrading strategies that improve bio-oil quality and/or reduce oxygen content are reported in the literature, including hydrogenation and hydrodeoxygenation (“HDO”). See generally Elliot et al., U.S. Pat. No. 7,425,657; Laurent et al., Study of the hydrodeoxygenation of carbonyl, carboxylic and guaiacyl groups over sulfided CoMo/γ-Al₂O₃ and NiMo/γ-Al₂O₃ catalysts. 1. Catalytic reaction schemes., Appl. Catal. A 109 77-96 (1994); and Mahfud et al., Hydrogenation of fast pyrolysis oil and model compounds in a two-phase aqueous organic system using homogeneous ruthenium catalysts, J. Mol. Catal. A: Chem. 264 227-236 (2007). Other strategies involve esterification of the bio-oils. See generally Tang et al., One-step hydrogenation-esterification of aldehyde and acid to ester over bifunctional Pt catalysts: A model reaction as novel route for catalytic upgrading of fast pyrolysis bio-oil, Energy Fuels. 22 3484-3488 (2008); Xu et al., Bio-oil upgrading by means of ethyl ester production in reactive distillation to remove water and improve storage and fuel characteristics, Biomass Bioenergy 21 1056-1061 (2008). In addition, selective extraction methodologies have been employed. See generally Deng et al., Green solvent for flash pyrolysis oil separation, Energy Fuels 23 3337-3338 (2009); Oasmaa et al., Solvent fractionation method with Brix for rapid characterization of wood fast pyrolysis liquids, Energy Fuels 22 (6) 4245-4248 (2008). In Tang et al., One step bio-oil upgrading through hydrotreatment, esterification, and cracking, Ind. Eng. Chem. Res. 48 6923-6929 (2009), upgrades to bio-oil in supercritical ethanol (T_{c}=240.9° C., P_{c}=63 bar) over a multifunctional Pd/SO₄⁻²/ZrO₂/SBA-15 catalyst at 280° C. and 20 bar H₂ partial pressure was reported (85 to 105 bar total pressure). Most of the reported improvements in the product quality (such as viscosity and density reduction, and increased pH and heating value) when using supercritical ethanol as a solvent appear to be due to esterification rather than deoxygenation of the bio-oils. A key limitation for many of the proposed bio-oil upgrading schemes is the requirement of high hydrogen partial pressures (up to about 300 bar) to enhance the intrinsic H₂ solubility in the liquid phase. Gas solubility in the liquid phase is inversely proportional to temperature, exacerbating this limitation at the high temperatures often required for HDO reactions. Thus, there remains a need to have an improved process for the deoxygenation of various oxygenates, such as those commonly found in bio-oils.

### BRIEF SUMMARY OF THE INVENTION

The present invention is directed to a process for the complete deoxygenation of an oxygenate. The process comprises the steps of forming a reaction mixture comprising the oxygenate, molecular hydrogen, and a hydrodeoxygenation catalyst in an inert solvent. The solvent is maintained at a reaction temperature that is 0.7 to 1.3 times the solvent critical temperature in absolute temperature units (K) and at a reaction pressure above the solvent critical pressure such that the molecular hydrogen and the oxygenate are miscible in the solvent. The deoxygenation process forms a completely deoxygenated product. The process is especially useful for the deoxygenation of aldehydes and other oxygenates found in bio-oils to produce corresponding alkanes.

In another aspect, the present invention is directed to a deoxygenation reaction mixture. The reaction mixture comprises the oxygenate, molecular hydrogen, a hydrodeoxygenation catalyst, and the inert solvent. The reaction mixtures is maintained at a temperature that is 0.7 to 1.3 times the solvent critical temperature in absolute temperature units (K) and at a pressure above the solvent critical pressure.

In yet another aspect, the present invention is directed to an improved process for the deoxygenation of an saturated aldehyde oxygenate having n carbon atoms. The process comprises the steps of forming a reaction mixture comprising the aldehyde oxygenate, molecular hydrogen, and the hydrodeoxygenation catalyst in an inert solvent. The reaction mixture is maintained at a reaction temperature that is 0.7 to 1.3 times the solvent critical temperature in absolute temperature units (K) and at a reaction pressure above the solvent critical pressure such that the molecular hydrogen and the oxygenate are miscible in the solvent. The deoxygenation process forms a completely deoxygenated product comprising a mixture of alkanes having n carbon atoms and n−1 carbon atoms. The selectivity of the alkane product can be tuned by altering the pressure of the system. In general, the selectivity of alkanes having n carbons is increased by increasing the pressure of the reaction mixture:

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 is a schematic representing the nonanal deoxygenation pathways for the experiments described in Example 1.

FIG. 2 illustrates the continuous fixed-bed reactor unit for supercritical deoxygenations described in Example 1.

FIG. 3 shows the isothermal pressure-tuning effects at near-critical temperature (1.13 T_{c}) on nonanal deoxygenation in n-hexane. T=300° C.; 2 g Pt/Al₂O₃; nonanal/H₂ molar ratio in feed=1:57; LHSV=8.60 mol/min. (▪) n-hexane as solvent, (□) n-hexadecane as solvent.

FIG. 4 shows the isothermal pressure-tuning effects at near-critical temperature (1.13 T_{c}) on space-time yield (STY), g of substrate converted*(g Pt⁻¹)*(h⁻¹). T=300° C.; 2 g Pt/Al₂O₃; nonanal/H₂ molar ratio in feed=1:57; LHSV=8.60 mol/min. (▪) n-hexane as solvent, (□) n-hexadecane as solvent.

FIG. 5 shows the isothermal pressure-tuning effects at near-critical temperature (1.13 T_{c}) on product selectivity. T=300° C.; 2 g Pt/Al₂O₃; nonanal/H₂ molar ratio in feed=1:57; LHSV=8.60 mol/min. (▪) octane, (♦) nonane, (▴) nonanol, (□) CO, (⋄)CH₄, (Δ) CO₂, (---) total hydrocarbons, (-) total liquids, (^{. . .} ) total gases.

FIG. 6 shows the isothermal pressure-tuning effects at near-critical temperature (1.13 T_{c}) on effective rate constants. T=300° C.; 2 g Pt/Al₂O₃; nonanal/H₂ molar ratio in feed=1:57; LHSV=8.60 mol/min. (▪) n-hexane as solvent, (□) n-hexadecane as solvent.

FIG. 7 shows the variation of diffusion coefficients for nonanal (solid line) and H₂ (dashed line) in n-hexane at 300° C. with pressure. Data valid for ρᵣ≧0.21 (22.3 bar for n-hexane).

FIG. 8 shows the effect of space velocity on effective rate constant at 30 bar and 300° C. nonanal/H₂ molar ratio in feed=1:57; 2 g Pt/Al₂O₃.

FIG. 9 is a comparison of the effective rate constant with gas-solid mass transfer the coefficient at near-critical temperature. T=300° C. (1.13 T_{c}); 2 g Pt/Al₂O₃; nonanal/H₂ molar ratio in feed=1:57; LHSV=8.60 mol/min.

FIG. 10 shows the temperature effect on nonanal deoxygenation. P=30 bar; 2 g Pt/Al₂O₃; nonanal/H₂ molar ratio in feed=1:57; LHSV=8.60 mol/min. (▪) Reaction conversion, plotted on left axis, (-) Nominal reactor temperature, plotted on right axis.

FIG. 11 shows the BET surface area of fresh, pretreated, and spent Pt/Al₂O₃ catalysts. Fresh=catalyst used as received; Pretreated=catalyst treated overnight at 300° C. and 100 sccm H₂; 30 bar (C16)=control experiment with n-hexadecane solvent; other data points are indicated by the reaction pressure used for the spent catalysts: T=300° C. (1.13 Tₐ); 2 g Pt/Al₂O₃; nonanal/H₂ molar ratio in feed=1:57; LHSV=8.60 mol/min.

FIG. 12 illustrates the selectivities for the experiments desired in Example 4 as a function of time.

FIG. 13 shows the conversion of nonanal as a function of time. Over 90% conversion was achieved in about two hours.

### DETAILED DESCRIPTION OF PREFERRED EMBODIMENT

The present invention relates a deoxygenation process which utilizes a solvent near or above supercritical conditions of temperature and pressure to facilitate catalytic deoxygenation of an oxygenate using molecular hydrogen. More specifically, the present invention is directed to a method for the deoxygenation of an oxygenate involving a reaction mixture comprising the oxygenate, molecular hydrogen, and a hydrodeoxygenation catalyst in a solvent maintained at or near supercritical conditions. In a preferred aspect, the reaction mixture is maintained at a reaction temperature that is preferably about 0.7 to 1.3 times the solvent critical temperature and at a pressure above the solvent critical pressure such that the molecular hydrogen and the oxygenate are completely miscible in the solvent. A homogeneous reaction mixture is thus formed in the fluid phase comprising the oxygenate and molecular hydrogen dissolved in the solvent. In a preferred aspect, the process provides for the complete deoxygenation of the oxygenate such that all oxygen is removed from the oxygenate to form a deoxygenated compound.

The present invention is well suited for the deoxygenation of the various oxygen-containing components of bio-oils. In the present invention, catalytic deoxygenation of the components of bio-oils in supercritical solvents can potentially eliminate the H₂ solubility limitation by bringing the reactants and molecular hydrogen into a single phase. In addition to eliminating interphase mass transfer resistances, the enhanced extraction of heavy hydrocarbons from the catalyst pores by the near-critical reaction mixture alleviates internal pore-diffusion limitations. The tunable properties (e.g., density and transport properties) of solvents in the near-critical region can have a dramatic impact on reaction selectivity. This is achieved by maintaining the reaction temperature (“Tᵣₓₙ”) near critical conditions—preferably about 0.7 to 1.3 times the solvent critical temperature, and still more preferably about 0.9 to 1.2 times the solvent critical temperature, wherein the temperature is in absolute temperature units (K). Thus, for example, in the present invention, Tᵣₓₙ may be about 0.7, 0.75, 0.8, 0.85, 0.9, 0.95, 1.0, 1.05, 1.1, 1.15, 1.2, 1.25, or 1.3 times the critical temperature of the solvent. The desired reaction temperature generally controls the choice of the supercritical solvent.

The term “oxygenate” as used herein denotes a hydrocarbonaceous compound that includes at least one oxygen atom. It is envisioned that the present invention may be employed to the deoxygenation of any oxygenate feed and is particularly directed to those of biological origin. Preferred oxygenates are the components of various bio-oils, which generally includes the product liquids from the pyrolysis of biomass. Bio-oils can be derived from plants such as grasses and trees, and other sources of ligno-cellulosic material, such as derived from municipal waste, food processing wastes, forestry wastes, and pulp and paper byproducts. Bio-oils can contain over 300 components of various oxygen-containing chemical functionalities, including aldehydes, hydroxyaldehydes, hydroxyketones, sugars, carboxylic acids, aromatics, and phenolics. See generally, Diebold et al., Additives to lower and stabilize the viscosity of pyrolysis oils during storage, Energy Fuels 11 1081-1091 (1997); Bridgwater, Production of high grade fuels and chemicals from catalytic pyrolysis of biomass, Catal. Today 29 285-295 (1996); Mohan et al., Pyrolysis of wood/biomass for bio-oil: a critical review, Energy Fuels 20 848-889 (2006); Furimsky, Catalytic hydrodeoxygenation, Appl. Catal. A: Gen. 199 147-190 (2000); Ingram et al., Pyrolysis of wood and bark in an auger reactor: physical properties and chemical analysis of the produced bio-oils, Energy Fuels 22 614-625 (2008). It is estimated that some bio-oils contain about 40 to 60% aliphatic oxygenates. Exemplary the oxygenates may contain 1, 2, 3, 4, 5, 6, 7, 8, 9, 10, 11, 12, 13, 14, 15, 16, 17, 18, 19, 20, 21, 22, 23, 24, 25, or more carbon atoms, or some range therebetween. For example, the oxygenate may contain five and twenty carbon atoms. Exemplary oxygenates are saturated or unsaturated aldehydes having 5 to 15 carbons, such pentanal, hexanal, heptanal, octanal, nonanal, decanal, undecanal, dodecanal, tridecanal, tetradecanal, pentadecanal, and the like. Other suitable oxygenates include saturated and unsaturated carboxylic acids, such as pentanoic acid, hexanoic acid, heptanoic acid, octanoic acid, nonanoic acid, decanoic acid, undecanoic acid, dodecanoic acid, and the like. Exemplary aromatic oxygenates include phenol, o-cresol, m-cresol, 3-ethylphenol, 2-tert-butylphenol, guaiacol, guaiethol and 2-isopropoxyphenol. The concentration of the oxygenate in the reaction mixture is typically about 0.05 to 1.0 mol/L for example, about 0.10, 0.2, 0.3, 0.4, 0.5, 0.6, 0.7, 0.8, 0.9, 1.0 mol/L, or some range therebetween.

The present invention also utilizes one or more metal hydrodeoxygenation catalysts in the reaction mixture. In one aspect, a Group VIB or Group VIII active metal catalyst is employed, especially those comprising nickel, cobalt, palladium, platinum, ruthenium, and rhodium, or mixtures thereof. The catalyst used in the present invention is preferably one containing one or more noble metals (Ru, Rh, Pd, Ag, Os, Ir, Pt, Au). Other suitable catalysts include copper based catalysts, such as copper-chromium or copper-zinc type. An exemplary catalyst is Pt/Al₂O₃.

The solvent used in the present invention is preferably an inert hydrocarbon having a critical temperature greater than about 400 K. Examples include saturated hydrocarbon having about 3 to 7 carbon atoms. Most preferred solvents are straight chain or branched alkanes or cycloalkanes having about 3 to 7 carbon atoms. Exemplary solvents include, but are not limited to n-hexane (T_{c}=507.6 K, P_{c}=30.2 bar), 2-methylpentane (isohexane, T_{c}=497.5 K, P_{c}=30.1 bar), 3-methylpentane (T_{c}=504.4 K, P_{c}=31.2 bar), 2,2-dimethylbutane (neohexane, T_{c}=488.8 K, P_{c}=30.8 bar), 2,3-dimethylbutane (T_{c}=499.9 K, P_{c}=31.3 bar), n-pentane (T_{c}=469.8 K, P_{c}=33.7 bar), 2-methyl butane (isopentane, T_{c}=460.4 K, P_{c}=33.8 bar), 2,2-dimethyl propane (neopentane, T_{c}=433.8 K, P_{c}=32 bar), butane (T_{c}=425.2 K, P_{c}=38 bar), 2-methyl propane (isobutane, T_{c}=408.1 K, P_{c}=36.5 bar), n-heptane (T_{c}=540.3 K, P_{c}=27.4 bar), cyclopentane (T_{c}=511.7 K, P_{c}=45.8 bar), and cyclohexane (T_{c}=553.5 K, P_{c}=40.7 bar) and mixtures thereof. The solvent is such that the solvent critical temperature is close to the reaction temperature. As discussed above, in general, the reaction mixture is maintained at a reaction temperature that is preferably about 0.7 to 1.3 times the solvent critical temperature, and still more preferably about 0.9 to 1.2 times the solvent critical temperature. Thus, it will be appreciated that the use of carbon dioxide by itself as a solvent is not within the scope of the present invention its relatively low critical temperature (T_{c}=304.4 K, P_{c}=73.8 bar). However, mixtures of carbon dioxide with the aforementioned inert hydrocarbon solvents may still be used in accordance with the present invention. That is, carbon dioxide may be used as a suitable co-solvent with the inert hydrocarbon solvent.

In a preferred aspect, the amount of molecular hydrogen used in the reaction mixture is in excess of the stoichiometric requirement for the catalytic hydrodeoxygenation of the oxygenate. Typically, the partial pressure of hydrogen in the reaction mixture is about 5 to 55 bar—e.g., about 5, 10, 15, 20, 25, 30, 25, 40, 45, 50, or 55 bar or some range therebetween.

In general, the deoxygenation conditions of the present invention are controlled by adjusting one or more of the following: the reaction temperature and pressure; the feedstock composition; the feedstock flow rate, that is the Liquid Hourly Space Velocity (“LHSV”); the specific reactor configuration; and, the level and extent of catalyst regeneration and/or re-circulation.

The temperatures and pressures of the reaction mixture will depend upon the solvent used and the nature of the hydrodeoxygenation reaction. In one aspect, the deoxygenation process occurs at a temperature greater than about 250, 260, 270, 280, 290, 300, 310, 320, 330, 340, or 350° C. In another aspect, the temperature is in the range of about 250 to 350° C. In a preferred aspect, the reaction mixture is maintained at a reaction temperature that is preferably about 0.7 to 1.3 times the solvent critical temperature and still more preferably about 0.9 to 1.2 times the solvent critical temperature (in absolute temperature units, K). Likewise, the total reaction pressure is typically greater than about 20, 30, 40, 50, 60, 70, 80, 90, 100 bar, or some range therebetween. In another aspect, the total reaction pressure is about 20 to 100 bar, and more preferably about 30 to 70 bar, and still more preferably about 30 to 50 bar. In a preferred aspect, pressure is maintained above the solvent critical pressure.

The liquid hourly space velocity (“LHSV”) generally depends upon the conversion desired. Lower LHSV generally results in higher substrate conversions, and vice versa. Typically, the LHSV is about 0.1 to 10 mol/min, e.g., about 1, 2, 3, 4, 5, 6, 7, 8, 9, or mol/min or some range therebetween. As such, fractional conversions greater than 0.6, 0.7, 0.8, 0.9, and even 0.95 may be achieved.

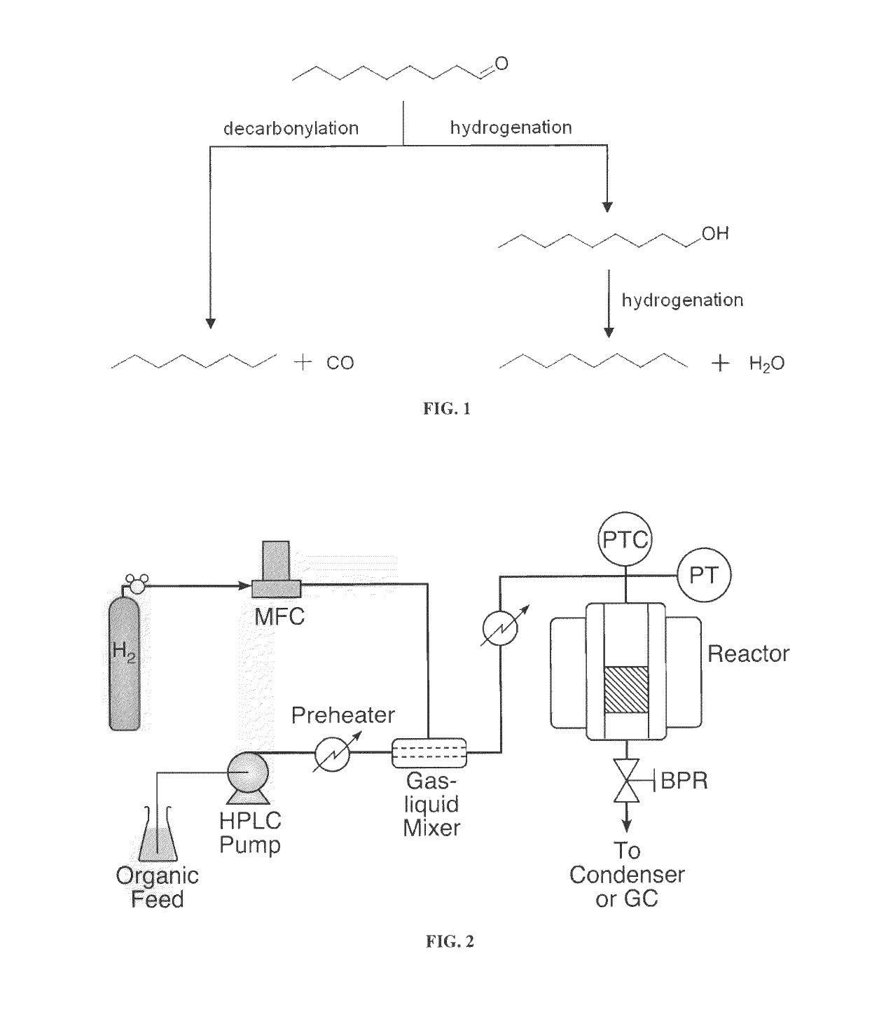

Without being bound by theory, deoxygenation of the oxygenates in the presence of hydrogen proceeds by a hydrodeoxygenation pathway (in which the oxygen in removed from the feedstock as water). In addition, for some oxygenates, the present invention may include a decarbonylation pathway that does involve hydrogen. For example, when the oxygenate is a saturated aldehyde having n carbon atoms (e.g., nonanal, n=9), the completely deoxygenated product comprises a mixture of corresponding alkanes having n carbon atoms (nonane, n=9) and n−1 carbon atoms (octane, n=8). The alkane having n carbons is produced via a hydrodeoxygenation pathway while the alkane having n carbons is produced via a decarbonylation pathway (see FIG. 1).

The deoxygenation of oxygenates in accordance with the present invention may be carried out in a variety of catalytic reactors, including fixed bed reactors, fluid bed reactors, slurry reactors, countercurrent free fall reactors, and concurrent riser reactors. Although any standard commercial scale reactor system can be used, including fixed bed or moving bed systems, it is preferable that the process is carried out in a dynamic bed system, and more preferably a dynamic bed system that is operable over a wide range of space velocities.

In the exemplary embodiment discussed in more detail below, the deoxygenation of an aliphatic oxygenate (the aldehyde nonanal) proceeds via both hydrogenation and decarbonylation pathways. As discussed more fully below, the nonanal and excess hydrogen were dissolved in supercritical0020hexane=234.45° C., P_{c}=30.2 bar) to form a single homogeneous phase and the reaction was studied in a fixed bed catalytic reactor. The deoxygenation occurred via simultaneous hydrodeoxygenation and decarbonylation to produce alkane products (octane and nonane) as generally shown in FIG. 1. This process results in steady catalyst activity and high throughputs (about 80 g nonanal converted*(g Pt)⁻¹*h⁻¹) over a broad range of pressures in the near-critical region.

The following examples are presented to demonstrate the present invention and to illustrate certain specific embodiments thereof. These examples should not be construed to limit the scope of the invention as set forth in the claims. There are many possible other variations, as those of ordinary skill in the art will recognize, which are within the spirit of the invention.

### Example 1

### Deoxygenation of Nonanal

In this example, n-hexane, n-hexadecane (99%), nonanal (95%), nonanol (97%), nonanc (99%), and octane (97%) were purchased from Fisher Scientific and used as received. Platinum on alumina pellets (1 wt %, 3.2 mm pellets) were purchased from Sigma-Aldrich. Hydrogen gas (industrial grade) was purchased from Airgas and used as received.

The catalytic deoxygenation reactions were performed in a continuous fixed-bed reactor (see FIG. 2) at 300° C. over supported Pt/Al₂O₃ catalysts. Specifically, the deoxygenation of nonanal is presented using supercritical n-hexane as the solvent. A solution of 0.3 mol/L nonanal in n-hexane was fed to the reactor using a Thermo Separation Products Constametric 3200 pump. Hydrogen was fed through a solenoid valve and metered into the reactor using a Brooks Model 5850E mass flow controller. The liquid was preheated to 250° C. prior to mixing with H₂ in an in-line mixer (Thar Designs). The reactor was heated by cartridge heaters (McMaster-Carr, 3618K193) and the temperature was maintained within ±5° C. via a sand bath. The temperature was measured with a profile thermoprobe (Omega, custom model) and the reactor pressure was controlled to ±0.35 bar with a back-pressure regulator (Circle Seal Controls, BP-60).

All electronic components were monitored and controlled using a data acquisition and control module (Measurement Computing, USB-2416-4AO) and LabVIEW version 8.6 software (National Instruments). The automated system allowed for temperature and pressure monitoring at multiple points along the flow path, proportional-integral-derivative control of the heaters and mass flow controller, precise control of the liquid pump flow, and automatic safety shutdowns in the event of an emergency or to prevent potentially hazardous conditions (excessive temperature or pressure).

Downstream from the reactor, the liquids were condensed via a cooling coil at 20° C. using a recirculating coolant pump (Neslab RTE-111) with a 50-50 water-antifreeze mixture. Liquid samples were drained from the bottom of the condenser and collected for 20 minutes and weighed to accurately measure the outlet volumetric flow rate. A sample of this collected liquid was then set aside for offline GC analysis.

The cooled gas stream from the condenser was plumbed directly to a GC for online analysis. A manual 3-way valve allowed switching between the GC and a vented line in order to measure gas flow rates. These flow rates were measured using a bubble flow meter. At least 5 measurements were performed for each sample to ensure accuracy.

Knowing the inlet concentrations and flow rates, and measuring the composition and flow rate of both the gas and liquid streams exiting the reactor, mass balance closure was obtained for the system. An example mass balance for the reaction is shown in Table 1. Control experiments conducted with only the n-hexane solvent, H2, and the Pt/Al2O3 catalyst confirmed that the solvent is inert under the experimental conditions. For this reason, carbon balances are reported on a hexane-free basis. The extents of mass balance closure (±10%) are within the expected experimental uncertainty.

TABLE 1
Typical C balance under steady state reaction conditions.

| | FEED | EFFLUENT |
|---|---|---|
| Compound | (kmol/min) | (kmol/min) |
| nonanal | 2.61 | 1.06 |
| nonanol | 0.00 | 0.21 |
| nonane | 0.00 | 0.09 |
| octane | 0.00 | 0.99 |
| CO | n/a | 0.14 |
| CO₂ | n/a | 0.01 |
| CH₄ | n/a | 0.05 |
| Total C, hexane-free basis | 2.61 | 2.56 |
| C difference, hexane-free basis | | 0.05 |
| C difference, hexane-free basis (%) | | 2.08% |

P = 30 bar; 2 g Pt/Al₂O₃; nonanal/H₂ molar ratio in feed = 1:57; LHSV = 8.60 mol/min
(n/a = not analyzed)

Gas phase products were analyzed online using an Agilent 5890 Series II gas chromatograph. The gas mixture is collected in two sampling valves. One valve sends the gas mixture through an Agilent HP-5 column (60 m×0.32 mm×0.25 μm) and to the flame ionization detector for analysis of hydrocarbons (C₁-C₉). The other valve directs the sample through two columns in series: a Restek 10% Rtx Stabilwax Silicoport W 100/120 followed by a 6′×2 mm SS Supelco 60/80 Carboxen 1000 (15′×⅛″ SS). The Carboxen 1000 column is the analytical column which separates the permanent gases. The Stabilwax column serves as a precolumn to prevent heavier (C₂+) compounds from reaching and perhaps damaging the analytical column. A valve switches during the method to back-flush these heavy compounds from the Stabilwax column to the vent. The temperature program for the analysis is as follows: (1) hold at 30° C. for 5 minutes, (2) ramp at 20° C./min to 210° C., (3) hold at 210° C. for 10 minutes. External standards of permanent gases as well as light hydrocarbons (Restek) were used to calibrate the response of the FID and the TCD detectors.

Liquid samples were analyzed offline using an Agilent 7890 gas chromatograph with an Agilent DB-FFAP column (30 m×0.30 mm×0.25 μm). The temperature program for the analysis is as follows: (1) hold at 100° C. for 2 minutes, (2) ramp at 10° C./min to 240° C., (3) hold at 240° C. for 10 minutes. External standards of nonanal, nonanol, nonane, and octane were used to calibrate the FID response.

BET analyses of fresh and spent catalysts were performed using a Micromeritics Gemini 2360 analyzer. Approximately 0.3 g of catalyst was loaded into a sample tube and dried at 90° C. under flowing nitrogen for one hour prior to analysis. The instrument method consisted of four equilibration cycles at 77 K (in liquid nitrogen) at various vacuum pressures with an evacuation rate of 300 mmHg/min. The BET surface area of the sample was calculated by the StarDriver software based on the volume (at STP) of nitrogen absorbed per gram of sample at each equilibration pressure.

Pressure-Tuning Effects

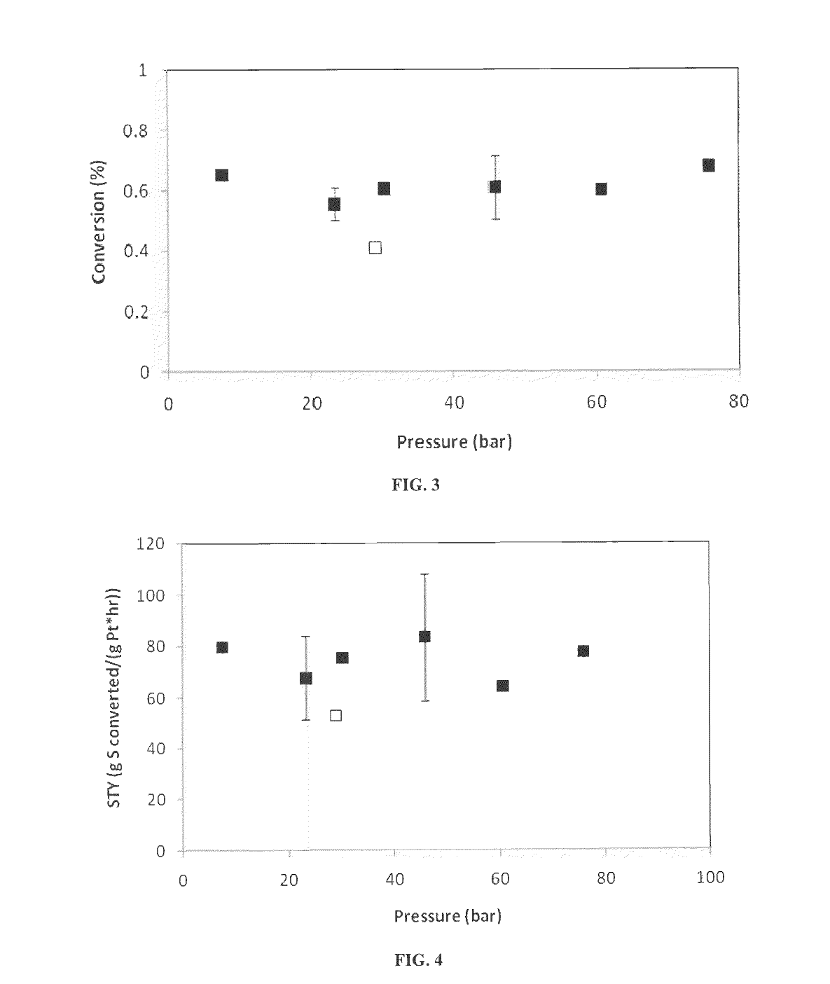

Experiments were conducted to examine the pressure effect on the reactions. All reactions were conducted at identical feed rates of substrates at 300° C. For a fixed molar feed rate (consisting of nonanal, hydrogen and hexane), the reactor pressure is controlled via the back pressure regulator in the exit stream. As shown in FIG. 3, total reactor pressure does not appear to affect the nonanal conversion. In the investigated pressure range, the nonanal conversion remains relatively constant at 0.61±0.05. A control experiment with hexadecane (C₁₆H₃₄), a liquid under the reaction conditions, was performed to demonstrate the advantages of supercritical hexane as solvent wherein the catalyst is exposed to a single phase that contains the dissolved reactants. With hexadecane, the nonanal conversion decreases to 0.41, suggesting reduced H₂ solubility in the reaction phase and/or poor gas/liquid transport across the wetted catalyst surface. These trends are also reflected, as expected, in, the lack of variation of space-time conversion [g of substrate converted*(g Pt)⁻¹*h⁻¹] with pressure (FIG. 4). The relatively steady space-time conversion values of about 80 g of substrate converted*(g Pt)⁻¹*h⁻¹ at mild supercritical pressures (tens of bars) are quite high.

At constant molar feed rates, the partial pressures of nonanal and H₂ increase proportionally with the total pressure, resulting in higher reactant concentrations at higher pressures. However, the reaction conversion (also space-time yield) shows no corresponding increase with pressure (i.e., concentration). The lack of any trend suggests that the overall conversion rate is controlled by external mass transfer limitations. Simple modeling based on this assumption supports this hypothesis (see Example 2 below).

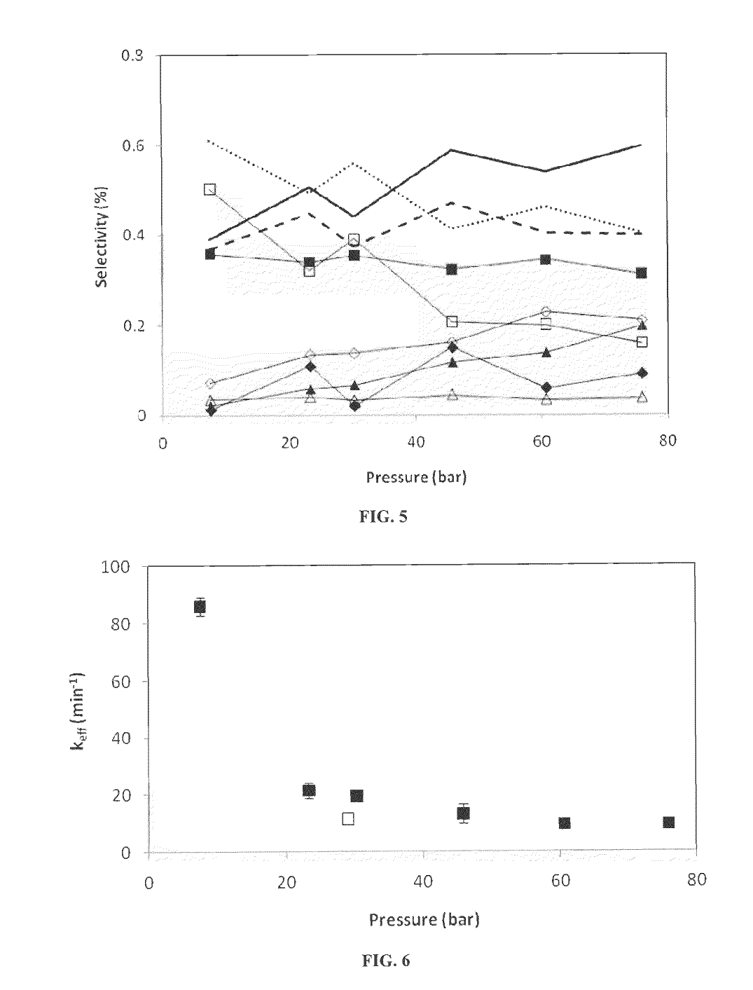

Although the reactor pressure does not affect the nonanal conversion, the product selectivity does appear to vary with the reaction pressure (see FIG. 5). In general, higher pressures result in enhanced selectivity to liquid products relative to permanent gases. Products from both the hydrogenation (nonanol, nonane) and the decarbonylation (octane, CO) pathways are present in the product stream, with the decarbonylation seemingly favored at all reaction conditions. Octane and carbon monoxide (CO) are produced in approximately equimolar amounts, as would be expected based on the decarbonylation reaction stoichiometry. Total selectivity to hydrocarbons (nonane and octane) remains relatively constant near 40% regardless of the reaction pressure, but decarbonylation appears to give way to hydrogenation at higher pressures, suggesting that higher H₂ partial pressures may favor the hydrogenation pathway. Selectivity to methane also increases with pressure, possibly suggesting an alternate methanation pathway based on higher CO and hydrogen pressures.

Temperature Effect

Additional experiments were undertaken to study the effect of temperature on the reaction rates. The experiment was run at 30 bar with constant molar flow rates as given in the experimental section. The temperature was maintained at 300° C. for the first four hours, and then decreased to 280° C. for four hours. After temporarily stopping the reactant flow and treating the catalyst at 300° C. and 100 sccm H₂ overnight, the reaction was resumed and performed for four hours at 320° C. followed by four hours at the original reaction conditions at 300° C. As shown in FIG. 10, the temperature effect on the reaction appears to be very mild. All conversions remain within the 0.61±0.05 window established in the pressure effect experiments. Product distribution data (not shown) also show no noticeable temperature dependence. These results again support the hypothesis that the reaction is mass-transfer limited. A kinetically controlled reaction should show strong temperature dependence (with activation energy on the order of tens of kcal/gmol), while diffusion coefficients show a relatively weak temperature dependence.

### Example 2

### Modeling

A simple mathematical model was developed to better understand the underlying physicochemical processes of Example 1. The model is based on an integral packed bed reactor mole balance assuming that the reaction is isothermal and that the lumped overall nonanal conversion rate is pseudo-first order in nonanal concentration. This assumption is valid given the 57:1 molar ratio of H₂:nonanal in the feed and the fact that the H₂ concentration did not significantly decrease in the product stream. The resulting model equation is shown in Equation 1:

$${k}_{eff} = - {ρ}_{cat} \frac{υ}{W} ln ( 1 - {x}_{c 9} ) \qquad ( 1 )$$

Where k_{eff}=effective rate constant, min⁻¹; ρ_{cat}=catalyst packing density, g/mL; v=total volumetric flow rate at reaction temperature and pressure, mL/min; W=mass of catalyst, g; x_{C9}=nonanal conversion, dimensionless.

Based on the steady state nonanal conversion, effective rate constant values were estimated using equation 1. As shown in FIG. 6, the effective rate constant decreases exponentially with pressure implying the existence of external mass transfer limitations (if the reaction rates were controlled by intrinsic kinetics, then the isothermal rate constants would not be expected to vary in this pressure range). In such a case, the effective rate constant would be representative of the external mass transfer coefficient (k_{g}=D_{c}/δ) where D_{c} represents the diffusivity of reactants in the boundary layer and δ is the layer thickness. The diffusivity values would vary inversely with pressure from gas-like values at subcritical pressures (wherein the film properties are gas-like) to liquid-like values at sufficiently high supercritical pressures (at >2 P_{c} of n-hexane, the film attains liquid-like properties).

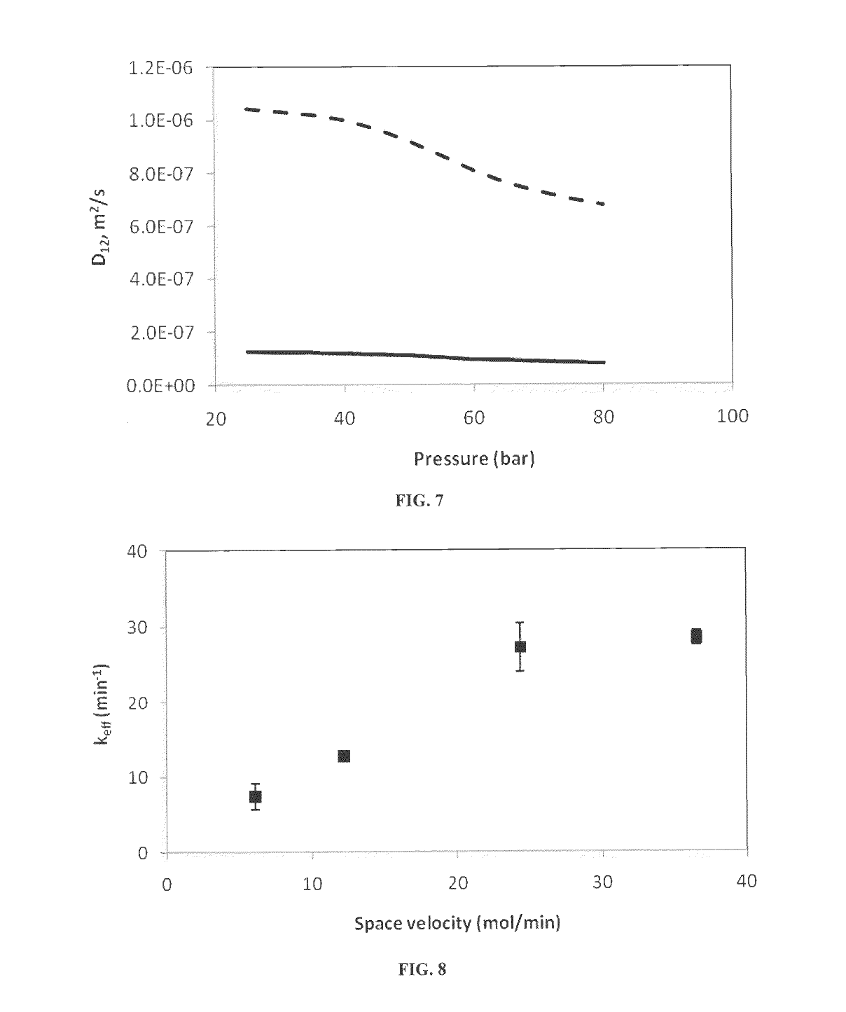

Diffusion coefficients in supercritical fluids at infinite dilution have been measured and modeled by a number of researchers. Using the correlation from He et al., Estimation of infinite-dilution diffusion coefficients in supercritical fluids, Ind. Eng. Chem. Res. 36 4430-4433 (1997), the predicted data for the diffusion coefficients of nonanal and H₂ in supercritical n-hexane are shown in FIG. 7. At higher pressures, the solvent transitions from gas-like to liquid-like diffusion properties, resulting in a decrease in the effective reaction rate constant under mass transfer controlled conditions. Note that at pressures above 60 bar (about 2 P_{c} of n-hexane), the diffusion coefficient becomes insensitive to pressure at liquid-like densities. Also note that under mass-transfer controlled conditions, the nonanal conversion rate is approximately equal to the product of the mass transfer coefficient and the concentration driving force (proportional to the reactor pressure). Since the mass transfer coefficient varies inversely with pressure, this product (i.e., the nonanal conversion rate) will be nearly constant with pressure as observed experimentally (FIG. 3). Experiments in which the space velocity was varied showed that the effective rate constants (at a constant P and T) increase with space velocity and asymptotically attain a constant value (FIG. 8). As inferred from FIG. 8, the space velocity corresponding to the experiments in FIG. 3 is clearly in the mass transfer limited region. In any future studies aimed at investigating reaction kinetics, the space velocity should be clearly higher to avoid external mass transfer limitations. Although the data reported herein are in the mass transfer controlled regime, the results nevertheless show that the supercritical deoxygenation process is viable and attractive.

Additional calculations were performed to estimate the gas-solid mass transfer coefficient, k_{g} (cm/s). The following correlation was used to relate the Sherwood number (“Sh”) to the Reynolds number (“Re”) and the Schmidt number (“Sc”):

$$Sh = \frac{{k}_{g} ρ}{G} = \frac{0.458}{{ɛ}_{B}} ( \frac{{a}_{t}}{{a}_{m}} ) {( Re )}^{- 0.41} {( Sc )}^{- 2 / 3} \qquad ( 2 )$$

Where ρ=fluid density, g/cm³; G=mass velocity of the fluid, g/cm²/s; ε_{B}=bed void fraction; aₜ=total surface area of the particles, cm²; aₘ=area of mass transfer available, cm²; Re=dₚ Glμ; dₚ=average particle size, cm; μ=fluid viscosity, g/cm*s; Sc=μρ⁻¹D₂₁⁻¹; D₂₁=molecular diffusivity of nonanal (limiting reactant) in n-hexane, cm²/s; k_{g}=gas-solid mass transfer coefficient, cm/s.

The following assumptions were made to simplify this approximation: aₜ=aₘ, ε_{B}=0.35 for cylindrical pellets with dₚ˜0.1 dₜ (tube diameter, cm) based on McCabe, W. L.; Smith, J. C.; Harriott, P., Unit Operations in Chemical Engineering 5th ed. McGraw-Hill, New York, p 1130 (1993); and dₚ=0.34 cm based on the equivalent diameter for a cylinder. In addition, k_{g} was converted to cm/min and multiplied by the effective particle surface area, aₚ=6 (1−ε_{B})/dₚ, to generate a product in units of cm⁻¹.

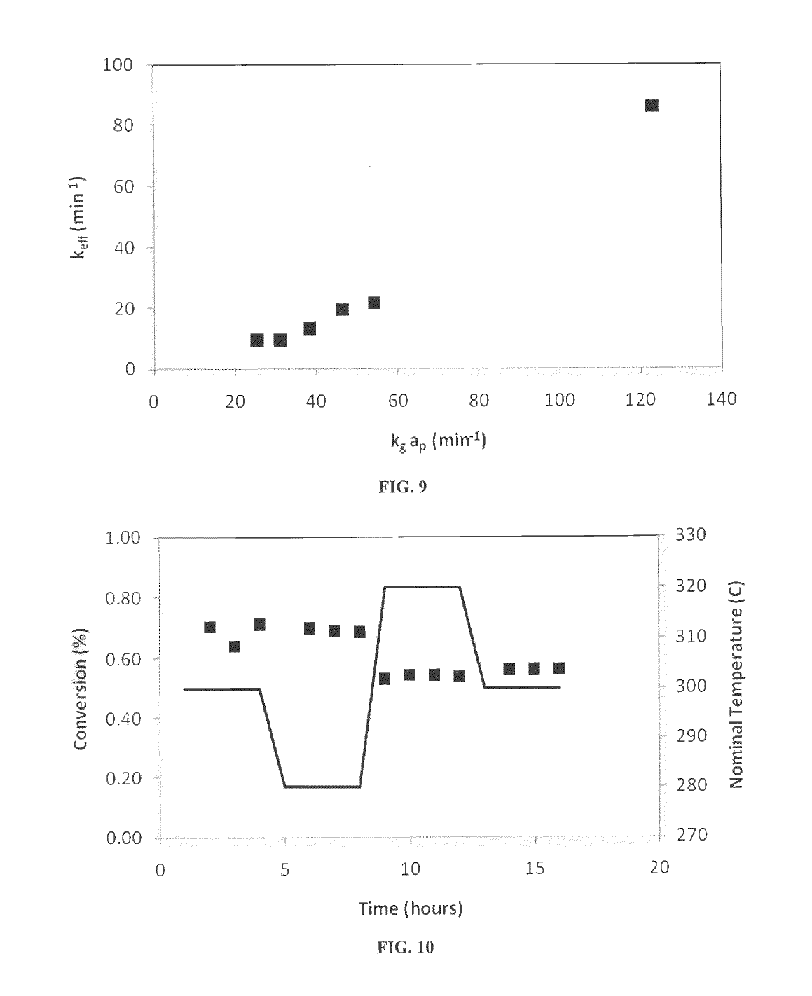

FIG. 9 compares the effective rate constants, k_{eff}, to the predicted gas-solid mass transfer coefficients (k_{g} aₚ). As can be seen in FIG. 9, the k_{g} aₚ values are of the same order of magnitude as the k_{eff} values estimated from the experimental data and the packed-bed reactor model. The linear relationship between these two quantities is further evidence that the deoxygenation of nonanal in supercritical hexane under the conditions in this study is controlled by external mass transfer of nonanal to the catalyst.

In sum, under the conditions studied, the reactions are mass transfer controlled as evidenced by the lack of pressure (i.e., concentration) effect on conversion. Also, the effective rate constant shows an exponential decrease with increasing pressure, which follows the predicted trends in the diffusion coefficients. Furthermore, mass transfer coefficients, predicted using published correlations, are of the same order of magnitude as the effective rate constants.

### Example 3

### Catalyst Characterization

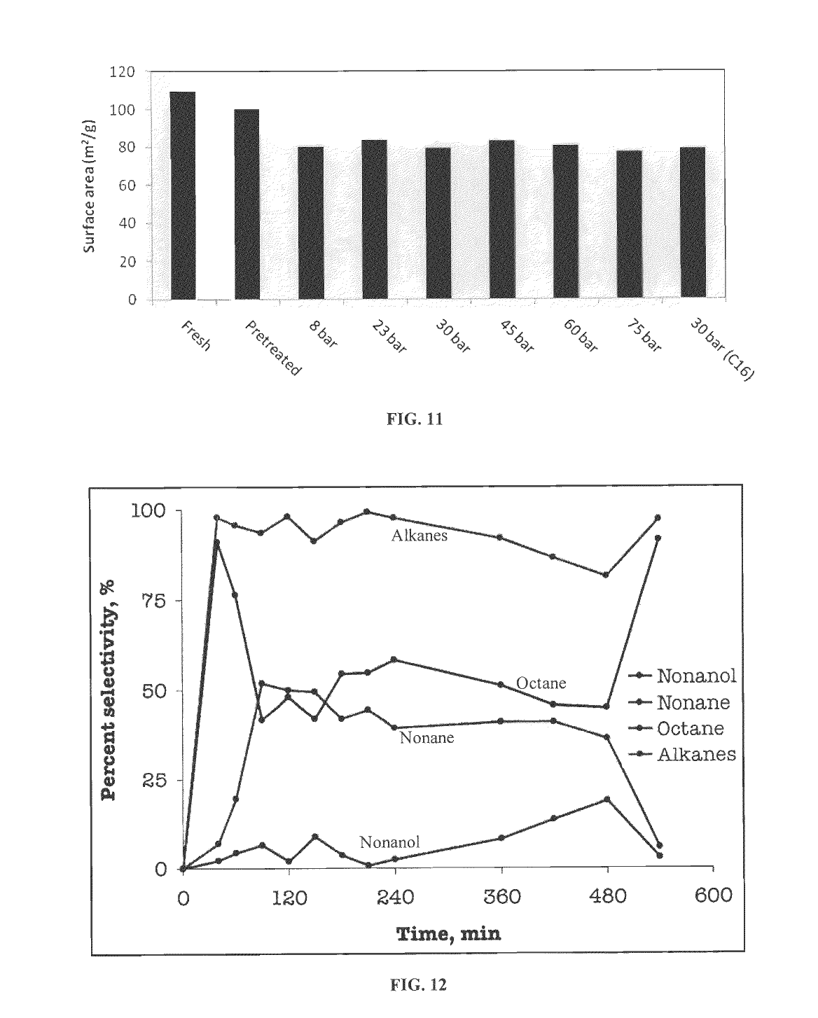

The BET surface area analyses of the fresh and spent Pt/Al₂O₃ catalysts are presented in FIG. 11. The surface area for fresh catalyst is 109.79 m²/g, and pretreating at 300° C. with 100 sccm H₂ reduces this surface area to 100.35 m²/g. Following reaction, the surface area for the spent catalysts is relatively constant at approximately 80 m²/g (±5%) for all reaction pressures examined. This trend holds true also when n-hexadecane is used as a solvent. These data suggest that the reaction conditions under study result in limited catalyst surface area loss, corroborating the steady conversions and selectivity corresponding to the various runs. These results also validate the assumption of constant catalyst activity when modeling the steady state conversion data.

### Example 4

### Effect of LHSV

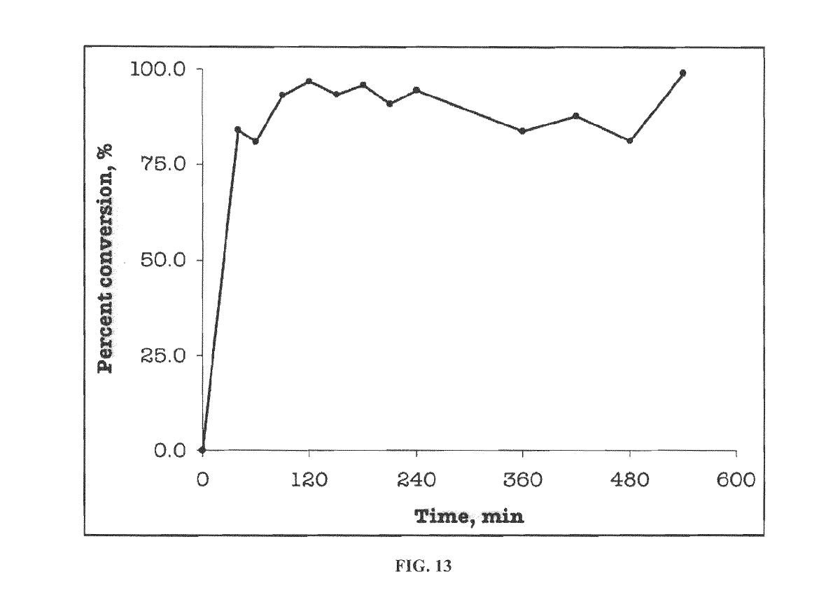

In this example, Pt/Al₂O₃-catalized deoxygenation of nonanal was investigated in a supercritical hexane in a fixed bed reactor at 300° C. and pressures of 39.2 bar with stoichiometric excess molecular hydrogen to produce nonane, nonanol, and octane, as well as light gases. The reaction included 2 g Pt/Al₂O₃ (0.5% Pt), a nonanal/H2 molar ratio in feed of 1:29, and LHSV=9.60 mol min-l. The space-time conversion was 218 g of substrate converted *(g Pt)⁻¹*h⁻¹. The product selectivities are illustrated in FIG. 12, and the conversion is shown in FIG. 13. High conversions of the substrate were achieved (greater than 90%) were achieved in less than two hours. High selectivity for nonane and octane was achieved.

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth or shown in the accompanying drawings are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A process for the complete deoxygenation of an oxygenate comprising:
forming a reaction mixture comprising said oxygenate, molecular hydrogen, and a hydrodeoxygenation catalyst in an inert solvent, said solvent having a solvent critical temperature and solvent critical pressure,
maintaining said reaction mixture at a reaction temperature that is 0.7 to 1.3 times the solvent critical temperature in absolute temperature units (K) and at a reaction pressure above the solvent critical pressure such that said molecular hydrogen and said oxygenate are miscible in said solvent and wherein said deoxygenation process forms a completely deoxygenated product.

2. The process of claim 1 wherein said solvent is a saturated hydrocarbon having 3 to 7 carbon atoms.

3. The process of claim 2 wherein the solvent is selected from the group consisting of n-hexane, isohexane, 3-methylpentane, neohexane, 2,3-dimethylbutane, n-pentane, isopentane, neopentane, butane, isobutane, n-heptane and mixtures thereof.

4. The process of claim 3 further comprising carbon dioxide as a co-solvent.

5. The process of claim 1 wherein the reaction temperature is about 250 to 350° C.

6. The process of claim 1 wherein the reaction pressure is about 20 to 100 bar.

7. The process of claim 1 wherein the partial pressure of the molecular hydrogen is in excess of the stoichiometric requirement for the catalytic deoxygenation of the oxygenate.

8. The process of claim 1 wherein the partial pressure of the molecular hydrogen is about 5 to 55 bar.

9. The process of claim 1 wherein the concentration of the oxygenate is about 0.05 to 0 mol/liter.

10. The process of claim 1 wherein the oxygenate contains five to twenty carbon atoms.

11. The process of claim 1 wherein the liquid hourly space velocity (LHSV) is about 0.1 to 10 mol/minute.

12. The process of claim 1 where the oxygenate is a bio-oil.

13. The process of claim 1 wherein the catalyst is a noble metal catalyst.

14. The process of claim 1 wherein the catalyst is Pt/Al₂O₃.

15. The process of claim 1 wherein the solvent, oxygenate, and molecular hydrogen are contacted with the catalyst in a fixed bed reactor in a continuous flow process and at a contact time sufficient to produce a deoxygenated product.

16. The process of claim 1 wherein the solvent, oxygenate, and molecular hydrogen are contacted with the catalyst in a fixed bed reactor in a continuous flow process and at a contact time sufficient to produce a deoxygenated product.

17. The process of claim 1 wherein said oxygenate comprises an saturated aldehyde, and wherein said solvent is a saturated straight chain or branched alkane having 3 and 7 carbon atoms, and wherein said reaction temperature is about 250 to 350° C., and wherein said reaction pressure is about 20 to 100 bar.

18. The process of claim 17 wherein said saturated aldehyde has n carbon atoms, and said completely deoxygenated product comprises a mixture of alkanes having n carbon atoms and n−1 carbon atoms.

19. The process of claim 1 wherein said reaction mixture is at temperature that is 0.9 to 1.2 times the solvent critical temperature in absolute temperature units (K).

20. A reaction mixture comprising:
an oxygenate;
molecular hydrogen;
a hydrodeoxygenation catalyst; and
and an inert solvent, said solvent having a solvent critical temperature and solvent critical pressure;
wherein said reaction mixture is at temperature that is 0.7 to 1.3 times the solvent critical temperature in absolute temperature units (K) and at a pressure above the solvent critical pressure.

21. The reaction mixture of claim 20 wherein said reaction mixture is at temperature that is 0.9 to 1.2 times the solvent critical temperature in absolute temperature units (K).

22. The reaction mixture of claim 20 wherein said oxygenate is an aliphatic aldehyde and said hydrodeoxygenation catalyst comprises platinum.

23. The reaction mixture of claim 20 wherein the solvent is selected from the group consisting of n-hexane, isohexane, 3-methylpentane, neohexane, 2,3-dimethylbutane, n-pentane, isopentane, neopentane, butane, isobutane, n-heptane and mixtures thereof.

24. The reaction mixture of claim 23 further comprising carbon dioxide as a co-solvent.

25. The reaction mixture of claim 20 wherein the reaction temperature is about 250 to 350° C.

26. The reaction mixture of claim 20 wherein the reaction pressure is about 20 to 100 bar.

27. The reaction mixture of claim 20 wherein the concentration of the oxygenate is about 0.05 to 1.0 mol/liter.

28. The reaction mixture of claim 20 wherein the oxygenate contains five to twenty carbon atoms.

29. An improved process for the deoxygenation of an saturated aldehyde oxygenate having n carbon atoms comprising
forming a reaction mixture comprising said aldehyde oxygenate, molecular hydrogen, and a hydrodeoxygenation catalyst in an inert solvent, said solvent having a solvent critical temperature and solvent critical pressure,
maintaining said reaction mixture at a reaction temperature that is 0.7 to 1.3 times the solvent critical temperature in absolute temperature units (K) and at a reaction pressure above the solvent critical pressure such that said molecular hydrogen and said oxygenate are miscible in said solvent and wherein said deoxygenation process forms a completely deoxygenated product comprising a mixture of alkanes having n carbon atoms and n−1 carbon atoms; and
increasing the selectivity of alkanes having n carbons by increasing the pressure in said reaction mixture.

30. The process of claim 29 wherein said solvent is a saturated hydrocarbon having 3 to 7 carbon atoms.

31. The process of claim 29 wherein the solvent is selected from the group consisting of n-hexane, isohexane, 3-methylpentane, neohexane, 2,3-dimethylbutane, n-pentane, isopentane, neopentane, butane, isobutane, n-heptane and mixtures thereof.

32. The process of claim 31 further comprising carbon dioxide as a co-solvent.

33. The process of claim 29 wherein the reaction temperature is about 250 to 350° C.

34. The process of claim 29 wherein the partial pressure of the molecular hydrogen is in excess of the stoichiometric requirement for the catalytic deoxygenation of the oxygenate.

35. The process of claim 29 wherein the partial pressure of the molecular hydrogen is about 5 to 55 bar.

36. The process of claim 29 wherein the concentration of the oxygenate is about 0.05 to 1.0 mol/liter.

37. The process of claim 29 wherein the oxygenate contains five to twenty carbon atoms.

38. The process of claim 29 where the aldehyde oxygenate is a bio-oil.

39. The process of claim 29 wherein the catalyst is a noble metal catalyst.

40. The process of claim 29 wherein the catalyst is Pt/Al₂O₃.

41. The process of claim 29 wherein said reaction mixture is at temperature that is 0.9 to 1.2 times the solvent critical temperature in absolute temperature units (K).
